(19) United States
(12) Patent Application Publication
(10) Pub. No.: US 2003/0175776 A1
(43) Pub. Date: Sep. 18, 2003

(54) Hybridization accelerator and accelerating method

(75) Inventors: Motonao Nakao, Tokyo (JP); Toshiki Morita, Tokyo (JP); Kanako Iwao, Tokyo (JP)

Correspondence Address: Reed Smith Hazel & Thomas LLP, Suite 1400, 3110 Fairview Park, Falls Church, VA, 22042-4503, US

(73) Assignee: Hitachi Software Engineering Co., Ltd.

(21) Appl. No.: 10/347,253

(22) Filed: Jan. 21, 2003

(30) Foreign Application Priority Data
Mar. 15, 2002 (JP) 71331/2002

(51) Int. Cl. C12Q 1/68; C12P 19/34; C12N 9/22

(52) U.S. Cl. 435/6; 435/199; 435/91.2

(57) ABSTRACT

Hybridization using a deoxyribonucleic acid chip is accelerated while strengthening bonding, and sensitivity and accuracy upon scanning is improved even after washing salts off. A deoxyribonucleic-acid-binding protein is used in a hybridization solution for a deoxyribonucleic acid chip so as to strengthen hybridization signal intensity. In addition, background noises are reduced by use of pure water in a washing process. As a result, a S/N ratio upon the hybridization can be improved.

### BACKGROUND OF THE INVENTION

1. Field of the Invention

The present invention relates to a hybridization accelerator which accelerates hybridization performed by use of a biochip, beads and the like in order to strengthen a complementary bond, to a method of accelerating hybridization, and to a method of detecting hybridization

2. Prior Art

Ionic polymers such as deoxyribonucleic acids (DNAs) or ribonucleic acids (RNAs) hybridize specifically with complementary strands thereof. Specific hybridization process is detected by labeling a complementary stand with a fluoro chrome or a radioisotope (RI) element.

Ionic polymers such as DNAs, RNAs, proteins (polyamino acids and polypeptides) possess charges where DNAs and RNAs possess negative charges attributable to phosphate groups, and where, proteins possess either positive charges or negative charges depending on types of amino acids therein. Double strands of a DNA normally cause repulsion attributable to negative ions, and therefore hybridization cannot take place in pure water. However, such hybridization is feasible in a system containing positive ions. For example, in a system containing ions such as Na⁺ ions, hybridization becomes feasible by hydrogen bonding attributable to chelating of Na⁺ ions into the phosphate groups of DNAs. Meanwhile, in the case of proteins and the like having positive charges, positively charged portions bond DNAs having negative charges. Accordingly, the progress of hybridization is frequently affected by an ionic strength.

A conventional hybridization system can perform hybridization only in a solvent with high ionic concentration because of the negative charges attributable to phosphate groups contained in DNAs and RNAs. For this reason, salts may separate out upon measurement after the hybridization, which may cause measurement errors. Moreover, the conventional hybridization system incurs a problem that the hybridized DNAs and the like may fall off upon washing with pure water.

Therefore, development of a technology has been long awaited for accelerating hybridization and for improving sensitivity and accuracy upon measurement, while strengthening complementary bonding by use of biochips, biobeads, and the like.

### SUMMARY OF THE INVENTION

As a result of extensive research, the inventors of the present invention have found that the aforementioned problems can be solved by use of a particular material as a hybridization accelerator, and have consummated the present invention accordingly.

Specifically, a hybridization accelerator of the present invention is a hybridization accelerator including a DNA-binding protein.

Moreover, a method of accelerating hybridization of the present invention includes a step of adding a DNA-binding protein to a hybridization solution.

The DNA-binding protein is characterized by possessing a positive charge in a DNA-binding domain thereof, and by bonding specifically or non-specifically to a DNA or an RNA which possesses a negative charge. Moreover, the double-stranded DNA bound by the DNA-binding protein exhibits a stronger bond as an ionic strength becomes lower.

The hybridization accelerator and the accelerating method of the present invention are applicable to various types of hybridization such as hybridization using a biochip or beads. In particular, the hybridization accelerator and the accelerating method of the present invention are applicable to the hybridization using a biochip or beads.

Timing to which the hybridization accelerator of the present invention is added can be selected from a wide range of timings. For example, the DNA-binding protein solution can be added to a biochip or beads before hybridization, or during the hybridization, and/or after the hybridization.

The hybridization accelerator and the accelerating method of the present invention aim at hybridization in which two or more ionic polymers complementarily bond to each other, such as hybridization for strengthening DNA-DNA complementary bonding, hybridization for strengthening DNA-RNA complementary bonding, hybridization for strengthening RNA-RNA complementary bonding, or hybridization for strengthening two ionic polymers.

Moreover, in a method of detecting hybridization of the present invention, a second ionic polymer (such as a second DNA) designed to bond complementarily to a first ionic polymer (such as a first DNA), which is fixed to a carrier, may be labeled with a fluoro chrome or a radioisotope (RI), so that residual fluorescence intensity may be measured after hybridization and washing. Otherwise, a fluorescent DNA-binding protein may be used therein or a DNA-binding protein may be colored, so that intensity of the fluorescence or the color may be measured later. In other words, the DNA-binding protein may be used as a hybridization accelerator and simultaneously as a labeling agent for measuring a result of the hybridization. When the DNA-biding protein is colored, such coloring may be carried out before the hybridization or after the hybridization. In the case of coloring the DNA-binding protein after the hybridization, the hybridization will be accelerated by adding the DNA-binding protein solution to the hybridization solution, and the result of the hybridization will be detected by coloring the DNA-binding protein specifically after the hybridization. On the other hand, in the case of coloring the DNA-binding protein before the hybridization, the DNA-binding protein will be labeled with a fluorochrome or a radioisotope (RI), and subsequently, the solution containing the labeled DNA-binding protein will be added to the hybridization solution to accelerate the hybridization. Then, after the hybridization and washing, the remainder of the fluorochrome or the radioisotope in the labeled DNA-binding protein will be measured for detecting the result of the hybridization specifically.

The DNA-binding protein to be used in the present invention refers to a protein having an affinity to a DNA and having a characteristic to bond a base sequence specifically or non-specifically. The DNA-binding protein mainly includes: (1) double-stranded DNA-binding proteins arranged to regulate gene expressions by modifying DNA structures; (2) single-stranded DNA-binding proteins required in the processes of replication, recombination and repair of DNAs; (3) proteins involved in retention of high-order structures of chromosomes; (4) proteins involved in the DNA-dependent ATP hydrolysis; (5) topoisomerases for forming DNA superhelix conformations; and the like. Most of the proteins in category (1) are transcription factors, and several structural motifs are known, such as the helix-turn-helix motif found out of structures of lambda phage Cro proteins and cAMP receptor proteins, the zinc finger motif in which a zinc ion chelated by cysteine and histidine, the leucine zipper motif formed in a zipper-like combination of two molecules of proteins each including leucine molecules that are aligned on one side of the α helix, or the like. The proteins in category (2) are proteins observed in various creatures from bacteriophages to higher organisms, and are referred to as SSB (i.e., single-stranded DNA-binding proteins). Histon protein included in a chromosome of a eukaryote is a typical example of the proteins in category (3), which forms a nucleosome structure. Formation of a nucleosome-like structure also observed in bacteria, in which similar HU proteins bond to each other. The proteins in category (4) includes proteins for DNA replication (such as DnaB proteins), proteins for recombination (RecA and RecBC proteins), and helicases which accelerate DNA unwinding. Furthermore, the present invention can apply not only the above-mentioned proteins possessing DNA-binding characteristics by nature, but also proteins which acquire DNA-binding characteristics due to physical or chemical processes.

As described above, the present invention can apply a wide range of DNA-binding proteins. In addition, the present invention includes not only the case of using one type of DNA-binding protein, but also the case of using two or more types of DNA-binding proteins in combination.

### BRIEF DESCRIPTION OF THE DRAWINGS

FIGS. 1(a) to 1(c) are schematic diagrams collectively showing an aspect of a DNA-binding protein accelerating hybridization.

FIG. 2 is a view showing spots on a biochip.

FIG. 3 shows images scanned after DNA hybridization in the case of adding the DNA-binding protein and in the case of not adding the DNA-binding protein.

### DETAILED DESCRIPTION OF THE PREFERRED EMBODIMENT

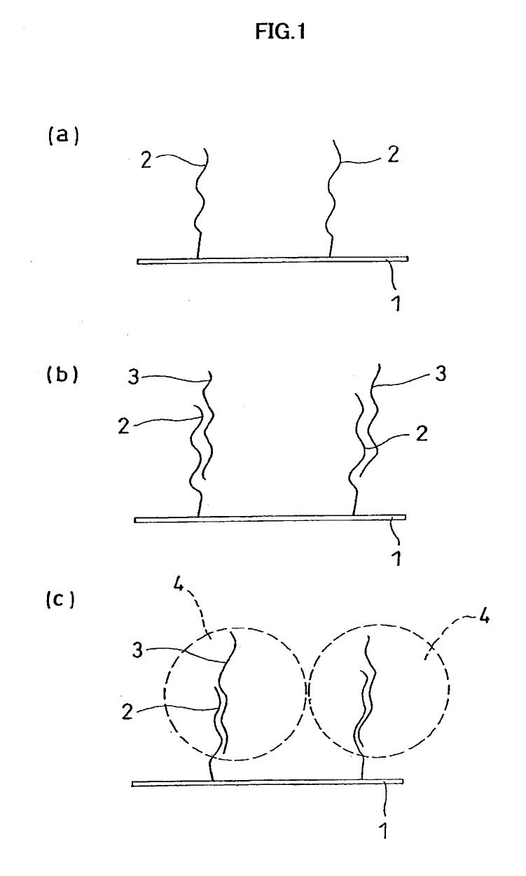

FIGS. 1(a) to 1(c) are schematic diagrams collectively showing the principle of a hybridization accelerator and a method of accelerating hybridization of the present invention. FIG. 1(a) shows a schematic diagram of a DNA chip for protein identification. A first DNA 2 having a specific base sequence is fixed to a carrier 1. If a second DNA 3 having a complementary base sequence to the base sequence of the first DNA is added to the DNA chip in an ionic solvent, then the both types of DNAs bond each other complementarily to form a double strand (FIG. 1(b)). If a DNA-binding protein 4 is added to the DNA chip, the protein 4 strengthens the double-strand bond in which the DNA 2 and the DNA 3 bond are complementarily to each other. Moreover, the protein 4 itself also bonds the double strand (FIG. 1(c)). Fluorescence intensity in such a phenomenon is measured and a sample DNA is thereby identified. Florescent labels may be given to the second DNA 3, or alternatively, may be given to the protein 4 beforehand or afterward. Fluorescence which remains after washing is scanned, and the hybridized DNA is identified based on the result of scanning.

The foregoing example has been described in the case of accelerating hybridization for strengthening DNA-DNA bonding. However, the present invention is not limited to the above-described case, but is widely applicable to various types of hybridization for ionic polymers.

### EXAMPLE

Now, an example of the present invention will be described. In this example, proteins which are already identified, namely, TRF, c-Myb, and Pur, are used as samples. Here, the TRF protein is disclosed in Nishikawa, T., Nagadoi, A., Yoshimura, S., Aimoto, S., and Nishimura, Y. “Solution structure of the DNA-binding domain of human telomeric protein, hTRF1. ” Structure, 6, 1057-1065 (1998); the c-Myb protein is disclosed in Ogata, K., Hojo, H., Aimoto, S., Nakai, T., Nakamura, H., Sarai, A., Ishii, S. & Nishimura, Y. “A helix-turn-helix-related motif with conserved tryptophans forming a hydrophobic core.” Proc. Natl. Acsd. Sci. USA, 89, 6428-6432 (1992); and the Pur protein is disclosed in Nagadoi, A., Morikawa, S., Nakamura, H., Enari, M., Kobayashi, K., Yamamoto, H., Sampei, G., Mizobuchi, K., Schumacher, M. A., Brennan, R. G. & Nishimura, Y. “Structural comparison of the free and DNA-Bound forms of the purine repressor DNA-binding domain.” Structure 3, 1217-1224 (1995).

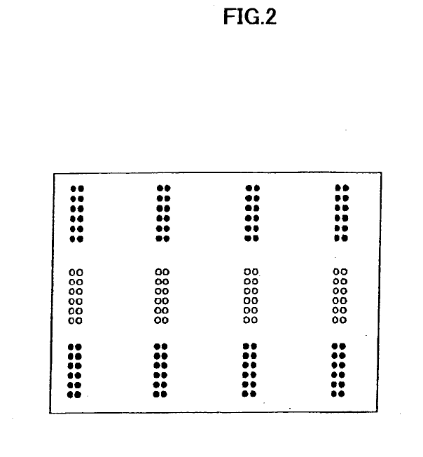

The following primers are firstly synthesized, and a DNA microarray is fabricated by spotting the primers as shown in FIG. 2 with a spotter.

Primer 1: 5′-GTTAGGGTTAGGG-3′ (in which biotin is introduced to the 5′ end)

Primer 2: 3′-CAATCCCAATCCC-540 (in which Cy5 is introduced to the 5′ end)

Primer 3: 5′-CGTAGAACTCCTCATCTC-3′ (in which biotin is introduced to the 5′ end)

Closed circles in FIG. 2 indicate Primer 1 and open circles indicate Primer 3. Then, hybridization was performed by preparing a 5×SSC solution with the concentration of 10 μm of Primer 2 as a sample DNA.

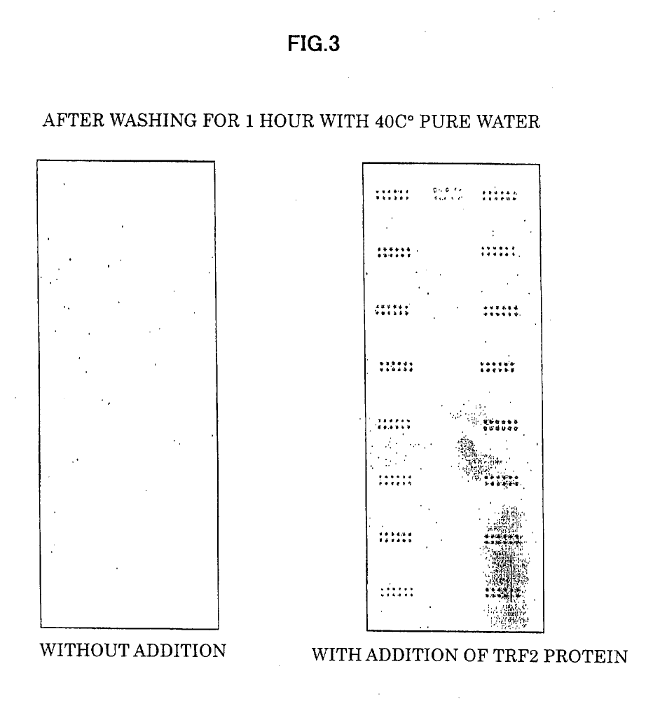

Thereafter, the TRF as the DNA-binding protein was added thereto and the DNA microarray was washed with pure water. A scanned image of the DNA microarray without addition of the TRF after washing with the pure water, and a scanned image of the DNA microarray with addition of the TRF after washing with the pure water are shown in FIG. 3.

As shown in FIG. 3, if the TRF is added to the DNA microarray, hybridizations signals remain distinct even after washing with the pure water.

In the past, it has been necessary to wash a DNA biochip after hybridization with an ionic solution containing a salt. However, detection of hybridization signals becomes feasible even after washing with pure water by use of a solution containing a DNA-binding protein. In other words, addition and use of a DNA-binding protein into a hybridization solution for a DNA chip or beads effectuates an increase in hybridization signal intensity, and use of pure water upon washing effectuates reduction in background noises. Eventually, an S/N ratio of the hybridization can be improved.

Whereas hybridization could not eliminate personal equation conventionally upon measurement, the present invention can easily standardize such hybridization.

What is claimed is:

1. A hybridization accelerator comprising:
a deoxyribonucleic-acid-binding protein.

2. The hybridization accelerator according to claim 1, wherein the hybridization accelerator aims at hybridization using any one of a biochip and beads.

3. The hybridization accelerator according to claim 1, wherein the hybridization accelerator aims at hybridization for strengthening a complementary bond between a deoxyribonucleic acid and another deoxyribonucleic acid.

4. The hybridization accelerator according to claim 1, wherein the hybridization accelerator aims at hybridization for strengthening a complementary bond between a deoxyribonucleic acid and a ribonucleic acid.

5. The hybridization accelerator according to claim 1, wherein the hybridization accelerator aims at hybridization for strengthening a complementary bond between a ribonucleic acid and another ribonucleic acid.

6. The hybridization accelerator according to claim 1, wherein the hybridization accelerator aims at hybridization for strengthening a bond between two ionic polymers.

7. The hybridization accelerator according to claim 1, wherein the deoxyribonucleic-acid-binding protein possesses fluorescence.

8. A method of accelerating hybridization comprising the step of:
adding a deoxyribonucleic-acid-binding protein solution to a hybridization solution.

9. The method of accelerating hybridization according to claim 8, wherein the method aims at hybridization using any one of a biochip and beads.

10. The method of accelerating hybridization according to claim 9, wherein the deoxyribonucleic-acid-binding protein is added to any one of the biochip and the beads in any event before the hybridization and during the hybridization.

11. The method of accelerating hybridization according to claim 9, wherein the deoxyribonucleic-acid-binding protein is added to any one of the biochip and the beads after the hybridization.

12. The method of accelerating hybridization according to claim 8, wherein the method aims at hybridization for strengthening a complementary bond between a deoxyribonucleic acid and another deoxyribonucleic acid.

13. The method of accelerating hybridization according to claim 8, wherein the method aims at hybridization for strengthening a complementary bond between a deoxyribonucleic acid and a ribonucleic acid.

14. The method of accelerating hybridization according to claim 8, wherein the method aims at hybridization for strengthening a complementary bond between a ribonucleic acid and another ribonucleic acid.

15. The method of accelerating hybridization according to claim 8, wherein the method aims at hybridization for strengthening a bond between two ionic polymers.

16. A method of detecting hybridization comprising the steps of:
labeling a second ionic polymer with any one of a fluoro chrome and a radioisotope, the second ionic polymer being designed to bond complementarily to a first ionic polymer fixed to a polymer chip carrier;
accelerating hybridization by adding a deoxyribonucleic-acid-binding protein solution to a hybridization solution; and
detecting a result of the hybridization by measuring any of the fluoro chrome and the radioisotope remaining after the hybridization and washing.

17. A method of detecting hybridization comprising the steps of:
accelerating hybridization by adding a deoxyribonucleic-acid-binding protein solution possessing fluorescence to a hybridization solution; and
detecting a result of the hybridization by measuring fluorescence intensity of the deoxyribonucleic-acid-binding protein after the hybridization and washing.

18. A method of detecting hybridization comprising the steps of:
accelerating hybridization by adding a deoxyribonucleic-acid-binding protein solution to a hybridization solution;
coloring the deoxyribonucleic-acid-binding protein specifically after the hybridization; and
detecting a result of the hybridization by measuring the colored deoxyribonucleic-acid-binding protein.

19. A method of detecting hybridization comprising the steps of:
labeling a deoxyribonucleic-acid-binding protein with any one of a fluoro chrome and a radioisotope;
accelerating hybridization by adding a solution containing the labeled deoxyribonucleic-acid-binding protein to a hybridization solution; and
detecting a result of the hybridization specifically by measuring any of the fluoro chrome and the radioisotope on the deoxyribonucleic-acid-binding protein remaining after the hybridization and washing.


SEQUENCE LISTING (3 sequences)

SEQ ID NO: 1 - DNA, 13 bp, Artificial Sequence
  Description of Artificial SequenceSynthetic DNA
gttagggttaggg

SEQ ID NO: 2 - DNA, 13 bp, Artificial Sequence
  Description of Artificial SequenceSynthetic DNA
caatcccaatccc

SEQ ID NO: 3 - DNA, 18 bp, Artificial Sequence
  Description of Artificial SequenceSynthetic DNA
cgtagaactcctcatctc